(19) United States
(12) Patent Application Publication
(10) Pub. No.: US 2024/0075256 A1
(43) Pub. Date: Mar. 7, 2024

(54) MULTI-LUMEN BALLOON CATHETERS SUITABLE FOR BALLOON OCCLUSION CHOLANGIOGRAPHY AND SPHINCTER OF ODDI DILATION AND RELATED METHODS OF USE

(71) Applicants: Wake Forest University Health Sciences, Winston-Salem, NC (US); Emory University, Atlanta, GA (US); Children’s Healthcare of Atlanta, Inc., Atlanta, GA (US)

(72) Inventors: Lucas Paul Neff, Winston-Salem, NC (US); Matthew S. Clifton, Atlanta, GA (US)

(21) Appl. No.: 18/262,226

(22) PCT Filed: Jan. 26, 2022
(86) PCT No.: PCT/US2022/013868

§ 371 Date: Jul. 20, 2023

Related U.S. Application Data
(60) Provisional application No. 63/263,572, filed on Nov. 4, 2021.
(60) Provisional application No. 63/142,140, filed on Jan. 27, 2021.

(51) Int. Cl. A61M 25/10; A61B 17/221; A61M 29/02

(52) U.S. Cl. CPC A61M 25/1011; A61B 17/221; A61M 29/02; A61B 2017/22038

(57) ABSTRACT

Multi-lumen catheters with first and second expandable members are configured so that one expandable member dilates the Sphincter of Oddi and the second anchors within the biliary duct distal to the Common Bile Duct/Cystic Duct junction or within the cystic duct. The multi-lumen catheters include a primary lumen sized and configured for delivering a flushing agent to remove gallstones together or separately with a dye to complete a Cholangiogram and also include at least one lumen in fluid communication with the first and second expandable members.

### RELATED APPLICATIONS

This patent application claims the benefit of and priority to U.S. Provisional Patent Application Ser. No. 63/142,140, filed Jan. 27, 2021, and U.S. Provisional Patent Application Ser. No. 63/263,572 filed Nov. 4, 2021, the contents of which are hereby incorporated by reference as if recited in full herein.

### FIELD OF THE INVENTION

The present invention relates to surgical devices suitable for gastroenterology.

### BACKGROUND

Choledocholithiasis is the presence of stones within the common bile duct. Bile stasis, bactibilia, chemical imbalances, increased bilirubin excretion, pH imbalances, and the formation of sludge are some of the factors which lead to the formation of these stones [1]. The incidence of choledocholithiasis has risen substantially in the last 3 decades and its management creates logistical and therapeutic challenges for surgeons [2-4]. It is noted that several publications and patent documents are cited in the background and specification of this patent application by footnote or journal reference. Each of these citations is incorporated herein by reference in their entirety as though set forth in full.

Choledocholithiasis can be managed safely by either open or laparoscopic cholecystectomy (LC) with pre-/post-operative endoscopic retrograde cholangiopancreatography (ERCP) or open or LC with intraoperative open common bile duct exploration (CBDE) or laparoscopic common bile duct exploration (LCBDE) [5-8]. LC with pre-/post-operative ERCP is a multistep process, while LC+LCBDE, for example, has the advantage of being a one-step, one-anesthetic process [9-11]. Despite this advantage, ERCP before or after cholecystectomy has firmly supplanted both CBDE and LCBDE nationwide [12]. This trend has persisted despite multiple reports of the efficacy and safety of LCBDE, specifically, and the associated decrease in hospital costs and length of stay [13-14].

One of the most significant factors contributing to the shift from LCBDE and to ERCP is believed to be the associated learning curve of the techniques involved in LCBDE, and in particular those involved in common duct clearance and the real/perceived logical hurdles at the time of laparoscopic cholecystectomy and the myriad device and technique options. Addressing those challenges may allow for broader adoption of LCBDE and be a benefit to patients.

### SUMMARY

Embodiments of the present invention provide a treatment system that can be used with a single intrabody insertion of a single multi-lumen catheter and is configured to drive adoption of LCBDE by allowing for an efficient and relatively simple-to-implement intervention.

Embodiments of the present invention are directed to a multi-lumen catheter that includes longitudinally spaced apart first and second expandable members. The first expandable member is sized and configured for dilating the Sphincter of Oddi and the second expandable member is sized and configured for anchoring the catheter anywhere in the biliary tree to create different size seals such as within the biliary duct distal to the Common Bile Duct/Cystic Duct junction or within the cystic duct. The multi-lumen catheter has a lumen configured for delivering a fluid for flushing to remove gallstones, together or separately with a dye, to complete a Cholangiogram.

Embodiments of the present invention are directed to related methods of treatment using the multi-lumen catheter.

Embodiments of the present invention are directed to a multi-lumen catheter that includes: a catheter body having opposing proximal and distal end portions; a first expandable member on the distal end portion; a second expandable member on the distal end portion, longitudinally spaced apart from the first expandable member; and a primary lumen that extends longitudinally and that is open at opposing proximal and distal ends thereof. The distal end of the primary lumen resides distally of the first and second expandable members. The multi-lumen catheter also includes at least one additional lumen in fluid communication with the first and second expandable members to controllably inflate and deflate the first and second expandable members.

The second expandable member can be distal of the first expandable member and can have a longitudinally extending length that is less than a length of the first expandable member.

The first expandable member can have/be formed of/a material with a first durometer. The second expandable member can have/be formed of/a material with a second durometer. The first durometer can be greater than the second durometer whereby the first expandable member can be configured to have less flexibility than the second expandable member thereby providing a non-compliant body suitable for dilating a sphincter or other target tissue.

The first expandable member can have a fully inflated state defining an outer diameter. The second expandable member can have a fully inflated state defining an outer diameter. The outer diameter of the first expandable member can be less than the outer diameter of the second expandable member in respective fully inflated states.

A length of the first expandable member can be 2-4 times greater than a length of the second expandable member.

The first expandable member can be closely spaced apart from the second expandable member by a longitudinal distance that can be in a range of 0.5 mm-20 mm.

The multi-lumen catheter can also include valves upstream of the first and second expandable members in fluid communication with the at least one additional lumen. The first and second expandable members can be independently/separately inflatable and deflatable.

The catheter body can be sized and configured for placement through a transductal opening in a biliary tree and the distal end portion can be sized and configured to distally terminate and be able to slidably extend into and retract from a duodenum of a patient.

The primary lumen can be coupled to a syringe comprising liquid and can be configured to allow liquid to pass through the distal end of the primary lumen into a duct of a patient.

The primary lumen can be configured to allow a medical device to be deployed through the primary lumen into the duct of the patient.

The deployable medical device can be an interventional wire.

The deployable medical device is a stone retrieval basket

The liquid can include saline.

The liquid can include a contrast agent.

The liquid can include a mixture of saline and a contrast agent.

The multi-lumen catheter can further include a plurality of longitudinally spaced apart radiopaque markings that can be positioned along the distal end portion of the catheter body thereby allowing visual indication of a location of one or both of the first and second expandable members in a medical image.

The first expandable member can have an elongate shape and can be made of a semi-rigid material. The second expandable member can have a spherical shape when inflated and can be formed of a material with increased flexibility relative to the first expandable member.

The first expandable member can be configured to expand to a diameter sufficient to dilate a target sphincter.

The diameter can be in a range of 1 to 20 mm.

The diameter can be in a range of 4 to 10 mm.

The multi-lumen catheter can further include a rotational inflation device coupled to a first lumen, the first lumen being provided by the at least one additional lumen. The first lumen can be in fluid communication with the first and/or second expandable member, typically the elongate first expandable member.

The second expandable member can be sufficiently compliant to expand to conform to and seal against surrounding tissue in apposition to walls of a duct in a biliary tree that it inhabits.

The second expandable member can be sized and configured to inflate to occlude a duct to facilitate pressurization and flushing of a distal portion of the duct.

The duct can be a biliary duct distal to a Common Bile Duct.

The duct can be a biliary duct distal to a Cystic Duct junction.

The duct can be a biliary duct within the cystic duct.

Other embodiments are directed to a method for using the multi-lumen catheter with the first and second expandable members. The methods include: a) advancing the multi-lumen catheter over a previously placed guidewire through a formed or natural orifice in a body of a patient toward a duodenum; b) at least partially inflating the first expandable member when the first expandable member is past a sphincter and in the duodenum; c) at least partially deflating and pulling back the first expandable member to traverse the sphincter; d) inflating the first expandable member to stretch the sphincter while the first expandable member straddles the sphincter; then e) deflating the first expandable member and pulling the multi-lumen catheter and guidewire together back in a proximal direction; then f) inflating the second expandable member to form a barrier in a duct; and g) discharging liquid from the multi-lumen catheter to flush debris in a distal direction out of a duct of a biliary tree.

The previously placed guidewire can be placed in a location that is provided from a transabdominal access site with a treatment path that enters into the biliary tree and distally terminates in the duodenum.

The inflation of the second expandable member to form the barrier in the duct can be within the biliary tree, and wherein the first expandable member can be at least partially deflated and reside outside the biliary tree or in the biliary tree when the second expandable member forms the barrier in the duct.

The discharged liquid can include saline.

The discharged liquid can include a contrast agent.

The discharged liquid can include a mixture of saline and a contrast agent(s).

The method can further include obtaining an X-ray of the biliary tree with the multi-lumen catheter in the patient after or during the discharging of the liquid.

The method can include deploying a medical device from a distal end of the multi-lumen catheter to dislodge or capture debris in a distal portion of the duct.

Yet other embodiments are directed to a biliary treatment system. The system include: a multi-lumen catheter comprising first and second expandable members on a distal end portion thereof. The multi-lumen catheter has a primary lumen and at least one additional lumen residing adjacent thereto and extending longitudinally. The distal end portion includes radiopaque markers. The system also includes a syringe with liquid coupled to the multi-lumen catheter and in fluid communication with the first and/or second expandable members and configured to inflate and deflate the first and/or second expandable members. The system also includes a Touhy-Borst connector coupled to the multi-lumen catheter and in communication with the primary lumen.

The syringe can be in fluid communication with only the second expandable member. The biliary treatment system can also include a rotational inflation device in fluid communication with the first expandable member to controllably inflate and deflate the first expandable member.

The biliary treatment system can also include a guidewire configured to be slidably received in the primary lumen.

The second expandable member can be distal of the first expandable member and can have a longitudinally extending length that is less than a length of the first expandable member.

The first expandable member can have/be formed of/a material with a first durometer. The second expandable member can have/be formed of/a material with a second durometer. The first durometer can be greater than the second durometer whereby the first expandable member is configured to have less flexibility than the second expandable member thereby providing a non-compliant body suitable for dilating a cystic duct and/or a sphincter.

The first expandable member can have a fully inflated state defining an outer diameter. The second expandable member can have a fully inflated state defining an outer diameter. The outer diameter of the second expandable member can be at least 50% greater than the outer diameter of the second first expandable member in respective fully inflated, operational, states.

A length of the first expandable member can be 2-4 times greater than a length of the second expandable member.

The first expandable member can be closely spaced apart from the second expandable member by a longitudinal distance that can be in a range of 0.5-20 mm.

The biliary treatment system can further include valves upstream of the first and second expandable members in fluid communication with the at least one additional lumen. The first and second expandable members can be independently/separately inflatable and deflatable.

The first expandable member can have an elongate shape and can be made of a semi-rigid material. The second expandable member can have a spherical shape when inflated and can be formed of a material with increased flexibility relative to the first expandable member.

The biliary treatment system can further include a medical device that is sized and configured to slidably deploy from a distal end of the multi-lumen catheter.

The first expandable member can be configured to dilate in a region of interest, such as a sphincter or cystic duct, in response to inflation pressure in a range of 1 atm to 10 atm to expand to an inflated state. The second expandable member can be configured to seal against walls of a duct in an inflated state having a larger outer diameter in response to inflation pressure that is lower than the inflation pressure applied to the first expandable member to inflate to one or more of its inflated operational inflated diameters and while the first expandable member is in at least a partially deflated state.

Still other aspects of the present invention are directed to a method for using the multi-lumen catheter with the first and second expandable members. The method includes: a) advancing the multi-lumen catheter over a previously placed guidewire through a formed or natural orifice in a body of a patient toward a duodenum; b) inflating the second expandable member to form a barrier in a duct in a biliary tree downstream of a cystic duct to thereby anchor the first inflatable member in the cystic duct; and c) inflating the first expandable member in the cystic duct to dilate the cystic duct.

The method can also include deflating the first and second expandable members, separately or concurrently, then withdrawing the catheter from the cystic duct, then inserting a choledochoscope into the dilated cystic duct to perform a medical procedure to thereby remove a stone or stones from the biliary tree.

Further features, advantages and details of the present invention will be appreciated by those of ordinary skill in the art from a reading of the figures and the detailed description of the preferred embodiments that follow, such description being merely illustrative of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF DRAWINGS

FIG. 1A is a top view of an example multi-lumen catheter according to embodiments of the present invention.

FIG. 1B is a top view of another example multi-lumen catheter according to embodiments of the present invention.

FIG. 2A is a top view of a surgical treatment system that includes the multi-lumen catheter shown in FIG. 1A with additional cooperating equipment according to embodiments of the present invention.

FIG. 2B is an enlarged view of a distal end portion of the multi-lumen catheter shown in FIG. 2A alongside a scale to illustrate example lengths and spacing according to embodiments of the present invention.

FIG. 2C is a lateral section view of an example lumen arrangement of the catheter body according to embodiments of the present invention.

FIGS. 3-13 are schematic illustrations of an example sequence of actions with respect to target intrabody anatomy that can be carried out using the treatment system with the multi-lumen catheter shown in FIG. 2A according to embodiments of the present invention.

FIG. 14 is a fluoroscopic image demonstrating the multi-lumen catheter of FIG. 1A with the second expandable/inflatable member in an inflated state, pulled back to access location and angle of the sphincter according to embodiments of the present invention.

FIG. 15 is a fluoroscopic image illustrating the multi-lumen catheter of FIG. 1A with the first expandable/inflatable member in an inflated state to dilate the sphincter—with correct placement indicated by the “waist” in the balloon as it comes up to profile (arrowhead) according to embodiments of the present invention.

FIG. 16 is an X-ray image of the multi-lumen catheter of FIG. 1A illustrating the distal second expandable/inflatable member (arrowhead) in an inflated state to fill the lumen of the Common Bile Duct and facilitate high pressure/volume flushing of the stones through the dilated sphincter according to embodiments of the present invention.

FIG. 17 is a medical image showing high pressure/volume flushing of stones and contrast through the dilated sphincter (arrow) after the guidewire is removed whereby contrast fills the duodenum (arrowhead) according to embodiments of the present invention.

FIG. 18 is a schematic illustration illustrating the treatment system employing a low-profile treatment device, such as an interventional wire, probe and/or basket, that can be deployed with, prior to, after or in the alternative of a flushing agent according to embodiments of the present invention.

FIG. 19 is a top view of an example treatment system comprising a deployable low-profile medical device that includes a basket according to embodiments of the present invention.

FIGS. 20A-20D are schematic illustrations of another sequence of actions or workflow that can be carried out using the multi-lumen catheter according to embodiments of the present invention.

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. The abbreviation “FIG.” may be used interchangeably with “Fig.” and the word “Figure” in the specification and figures. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in circuit of flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments. The term “about” means that the noted number can vary by +/−20%.

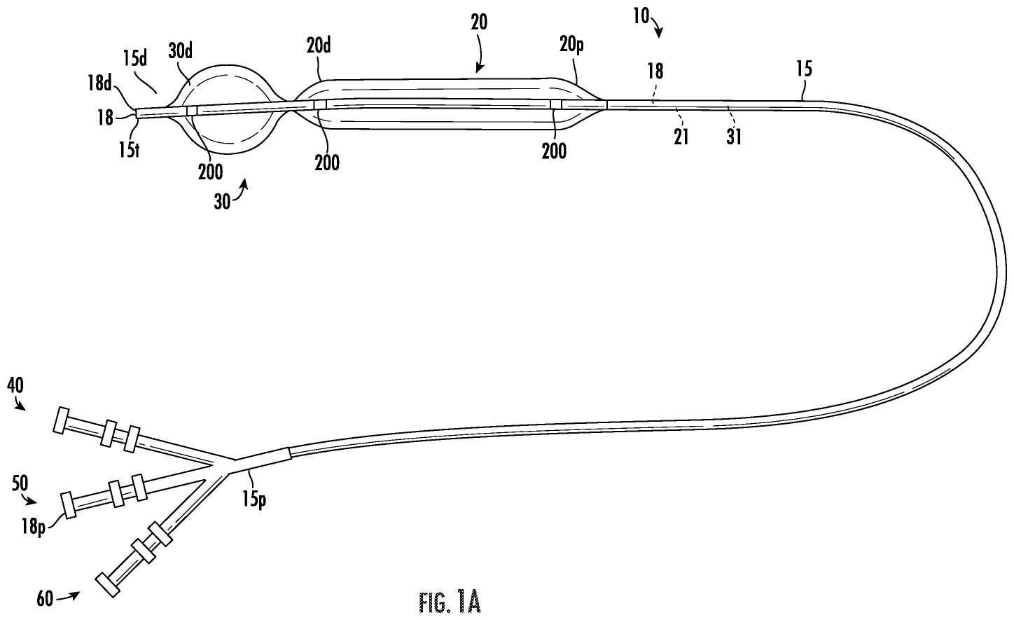

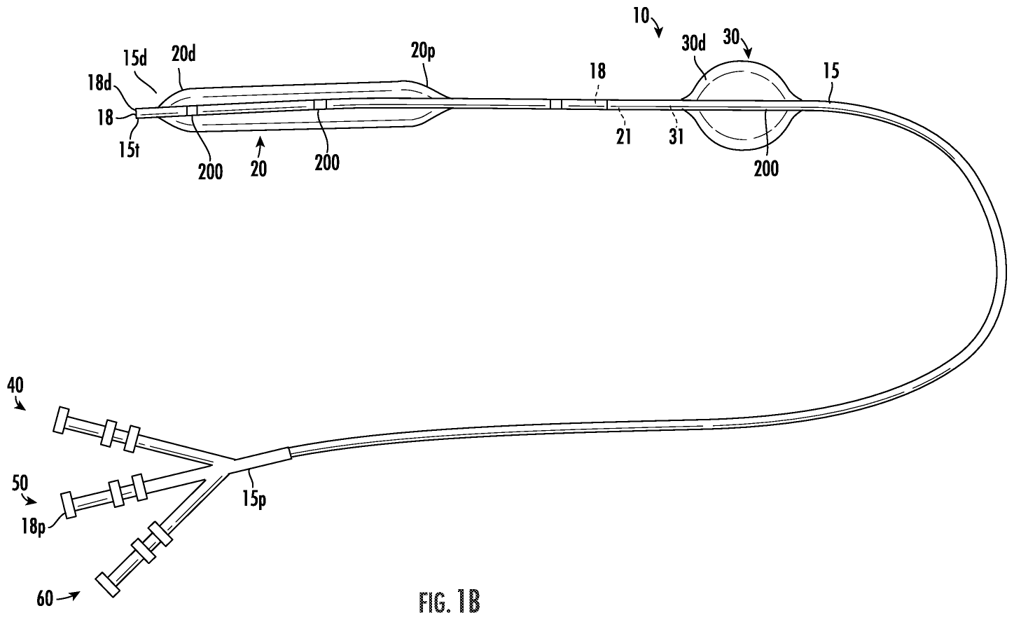

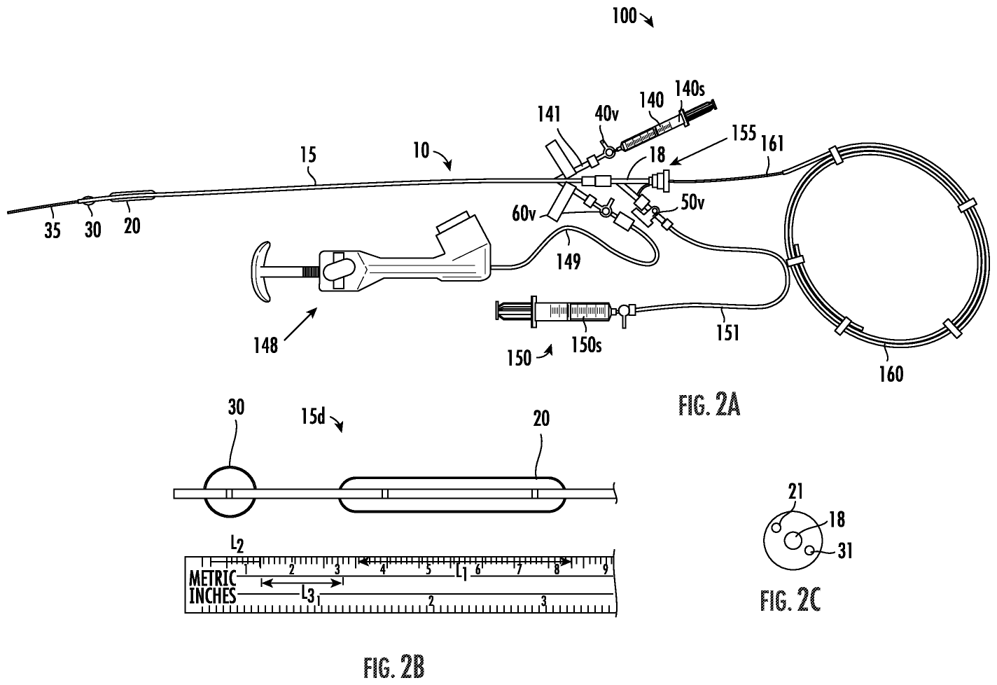

Referring to FIGS. 1A, 2A-2C an example multi-lumen catheter 10 is shown. The multi-lumen catheter 10 has a catheter body 15 with a primary lumen 18 that is open from a proximal end portion 15p through a tip 15t at the distal end portion 15d. The distal end portion 15d comprises a first expandable member 20 and a second expandable member 30. The expandable members 20, 30 can be interchangeably referred to as “inflatable members” which can be inflated to expand the expandable members 20, 30 using a fluid source such as a gas and/or a liquid.

As will be discussed further below, the multi-lumen catheter 10 can include a catheter body 15 with a proximal end portion 15p that comprises a plurality of connectors 40, 50, 60. Some or all of the connectors 40, 50, 60 can cooperate with fluid sources that together form a treatment system 100.

The catheter 10 can have a longitudinally extending length that is in a range of 50-80 cm. The catheter body 15 can have an outer diameter that is in a range of 4FR to 6FR. The catheter 10 can be suitable for human and veterinarian medical uses.

The catheter 10 can include one more radiopaque markers 200 on the distal end portion 15d of the catheter body 15. As shown, one of the one or more radiopaque markers 200 can reside at a distal end 20d of the first expandable member 20. As shown, one of the one or more radiopaque markers 200 can reside at a proximal end 20p of the first expandable member 20. One or more of the one or more radiopaque markers 200 can extend adjacent the second expandable member 30, shown as closer to a distal end 30d thereof than a proximal end thereof.

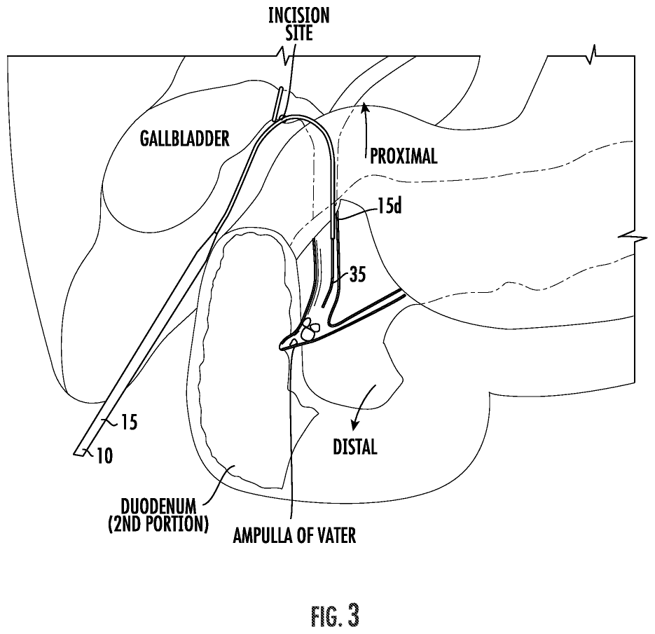

FIG. 1B illustrates the first expandable member 20 residing closer to the distal end portion 15d of the catheter body 15 than the second expandable member 30. This embodiment will result in a modification of the workflow/method steps described below with respect to FIGS. 3-13 and may be less efficient in certain uses relative to the embodiment shown in FIG. 1A.

FIG. 2A depicts connectors and fluid source components that are commercially available and can be attached to the multi-lumen catheter 10. As shown, the treatment system 100 includes a pressurized fluid source 140, shown as a syringe 140s, for selectively inflating/deflating the second inflatable member 30 (which can be a round compliant balloon). As also shown, the treatment system 100 also includes a Touhy-Borst Connector 155 that can be in communication with the primary lumen 18. The system 100 can also include a rotational inflation device 148 in fluid communication with the first inflatable member 20 for inflation/deflation thereof (e.g., a non-compliant elongate balloon). The treatment system 100 also includes a fluid source 150, shown as a contrast/saline flush injection syringe 150s, in fluid communication with the primary lumen 18.

The treatment system 100 can also include a guidewire 35, which can be for catheter guidance which may be configured with a floppy tip. However, other configurations of guidewires may be used. The guidewire 35 that has an outer diameter in a range of about 0.018-0.038 inches, more typically about 0.38 inches. The guidewire 35 can be slidably received through the primary lumen 18 and the connector 155.

Each connector 40, 50, 60 can be attached to a valve 40v, 50v, 60v, for selectively allowing fluid in or out of the lumen associated therewith. Extension conduits 141, 149, 151, 161 can also be used to form part of flow paths of the treatment system 100, some of which can be longer than others.

The multi-lumen catheter 10 can be configured for dilating the Sphincter of Oddi with the first expandable member 20 and anchoring a distal end portion of the catheter 10 in position with the second expandable member 30 at any location in the biliary tree such as within the biliary duct distal to the Common Bile Duct/Cystic Duct junction or within the cystic duct. The second expandable member 30 is configured to expand sufficiently and to different outer diameters to seal to tissue at those different locations. As shown in FIGS. 1A, 2A, the second expandable member 30 can be distal of the first expandable member 20. In other embodiments, as shown in FIG. 1B, the second expandable member 30 can be proximal of the first expandable member 20, e.g., the positions of the first and expandable members 20, 30 shown in FIG. 1A can be reversed.

The multi-lumen catheter 10 is configured to deliver a flushing agent(s) and/or one or more interventional devices (e.g., wire-based stone retrieval devices) to remove gallstones. The flushing agent(s) can be delivered via a lumen such as the primary lumen 18 together with a dye or the dye can be delivered separately to complete a Cholangiogram.

The primary lumen 18 may be configured as a central lumen 18. The primary lumen 18 has an open proximal end 18p coupled to connector 50 that branches to connector 155, adjacent the proximal end 15p of the catheter body 15. The multi-lumen catheter 10 can have at least one additional lumen 21, referred to as a second lumen 21. The second lumen 21 can extend longitudinally and can reside adjacent the primary lumen 18, in fluid communication with and configured to accommodate and deploy one or more of the expandable members 20, 30 attached to the distal end portion 15d of the catheter 10.

While the same lumen can be used to inflate/deflate each of the first and second inflatable members 20, 30, in other embodiments the multi-lumen catheter 10 can have dedicated separate lumens 21, 31 (FIG. 2C) for each inflatable member 20, 30. Thus, the multi-lumen catheter 10 can include a possible third lumen 31, which can longitudinally extend adjacent to the primary lumen 18 and/or the second lumen 21. The second lumen 21 can be in fluid communication and accommodate and deploy the first expandable member 20 and the third lumen 31 can be in fluid communication and accommodate and deploy the second expandable member 30.

The primary lumen 18 can slidably receive or accept a guidewire 35 to allow the multi-lumen catheter 10 to be advanceable thereover to a desired intrabody position. In use, the guidewire 35 can be placed in position in the body and the multi-lumen catheter 10 can then be advanced into place along the guidewire 35.

The primary lumen 18, or an additional lumen, can also be attached to a fluid source 150, such as a liquid filled syringe 150s, to allow liquid to pass through the distal end 18d of that lumen 18 (or the additional lumen) for deployment in the duct. The liquid can include a flushing agent comprising saline in order to flush the location of gallstones and other natural products. The flushing agent can comprise a contrast agent in order to opacify the biliary tree during diagnostic cholangiography.

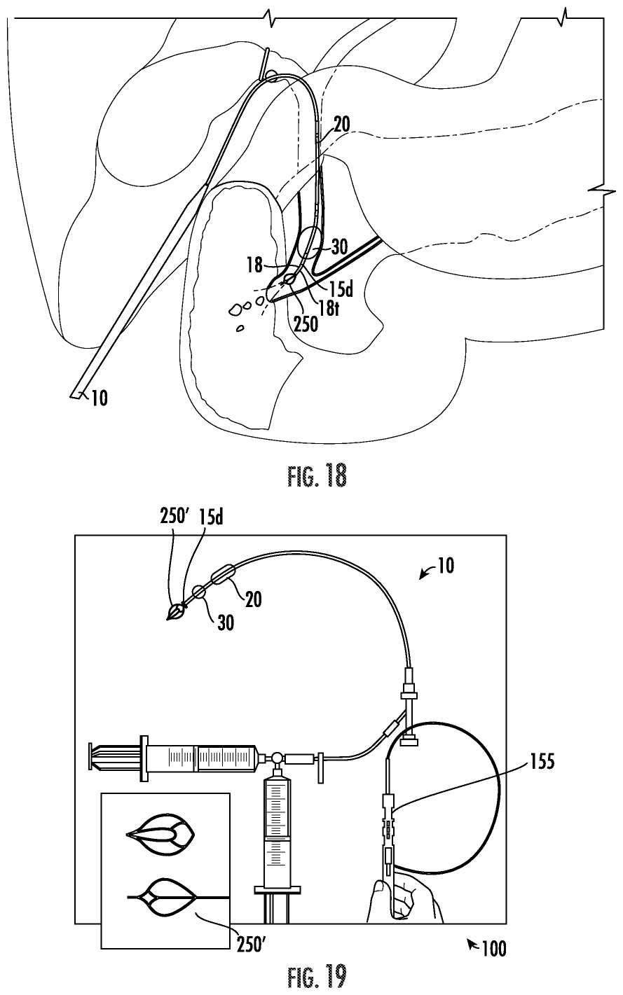

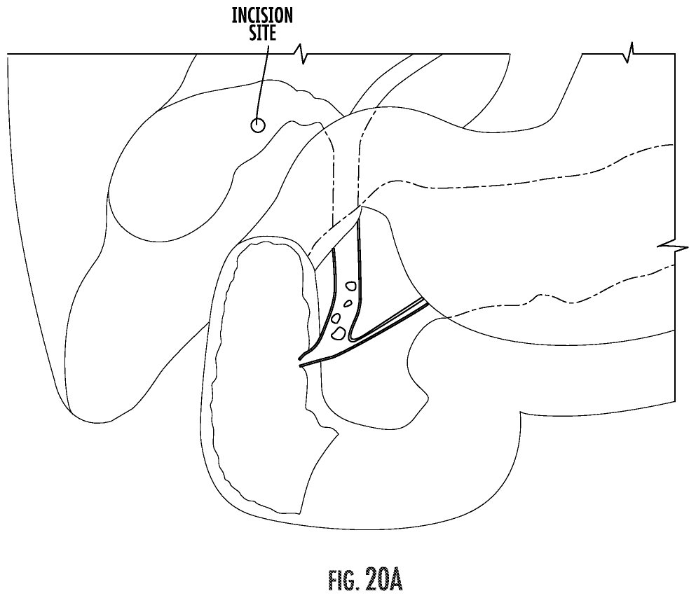

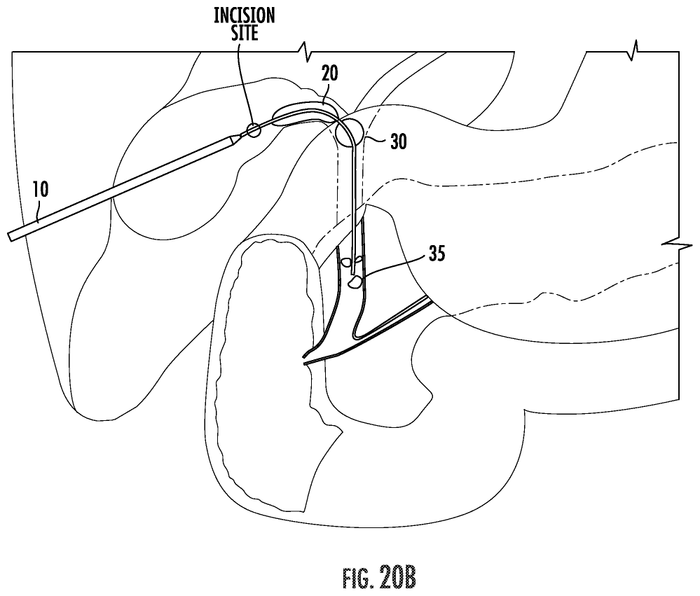

In some embodiments, the primary or central lumen 18 can be attached to a liquid filled syringe 150s and can also be configured to allow one or more other low-profile medical devices 250 (FIGS. 18, 19) to pass through the distal end 18d of the lumen 18 at the tip 15t of the catheter body 15 for deployment in the duct. The devices 250, 250′ can be inserted through the primary lumen 18 and can include, but are not limited to, an interventional wire, electrical or ultrasonic probe, a probe for laser lithotripsy, and/or a stone retrieval basket.

As discussed above, the second and third lumens 21, 31 (or lumina) can each be in fluid communication with and deploy a respective expandable member 20, 30. The second lumen 21 can be used to deploy the first expandable member 20. The first expandable member 20 can sometimes referred to as the “first”, “semi-flexible”, “elongated”, “non-compliant” or “semi-compliant” balloon. Sometimes terms are combined such as an “elongated, non-compliant expandable member.” It is attached to the distal end portion 15d of the catheter body 15 in fluid communication with the second lumen 21. In some embodiments, this first expandable member 20 has an elongate shape and is made from semi-flexible material. The first expandable member 20 can be non-compliant in order to move the tissue and fully inflate. The terms “semi-flexible” and “non-compliant” means that the first expandable member 20 has sufficient structural rigidity to be able to force a restriction in the sphincter to open when exposed to forces of the surrounding tissue. The first expandable member 20 can be elongate/elongated and be configured to expand using high pressure fluid to a target, controllable, outer diameter, such as to 6 mm, 8 mm, 10 mm, and the like. The first expandable member 20 can be in fluid communication with a high-pressure rotational inflation source 148 (FIG. 2A). The high-pressure inflation source 148 can provide inflation pressures of at least about 1 atm, such as inflation pressures in a range of about 1 atm to about 10 atm to inflate the first expandable member 20. In some embodiments, this first expandable member 20 is not limited to a semi-flexible material or an elongated shape. As will be appreciated by those of skill in the art, the first expandable member 20 can alternatively be configured as a semi-compliant (mid-pressure) balloon rather than a non-compliant (high-pressure) balloon in some particular embodiments.

The third lumen 31 can be configured to deploy the second expandable member 30. This second expandable member 30 is sometimes referred to as the “second”, “flexible”, “round”, or “compliant” balloon. Sometimes termed are combined. It can be attached to the distal end portion 15d of the catheter body 15 at the end of the third lumen 31. This second expandable member 30 can have a spherical shape (a round, 2-dimensional shape) and can be made from flexible material and/or a material that has a greater flexibility than the first expandable member 20. The second expandable or inflatable member 30 can be sufficiently compliant to open and conform to and/or seal against, the surrounding tissue and fully inflate in apposition to the walls of the duct that it inhabits. The second expandable member 30 can be inflated to a larger outer diameter than the first expandable member 20.

The second expandable member 30 can be inflated to a maximal outer diameter that is greater in size than a maximal outer diameter of the first expandable member 30 such maximal outer diameter can be generated using lower inflation pressures relative to the first expandable member 20, typically 40% or less inflation pressure. The lower pressures can be in a range of about 5 mmHg to about 200 mmHg, in some embodiments. The second expandable member 30 can be inflated/deflated using a syringe 140s (FIG. 2A) comprising fluid. The second expandable member 30 can be configured to inflate to a 6 mm-20 mm outer diameter, with 20 mm defining a maximal outer diameter, in some embodiments. The second expandable member 30 can inflate by volume rather than by pressure. The second expandable member 30 can (elastically) stretch by 100-800%, in some embodiments. In some embodiments, this second expandable member 30 is not limited to a flexible material or a spherical shape.

The first expandable member 20 can have a wall thickness that is greater than a wall thickness of the second expandable member 30. The first expandable member 20 can be formed of a material of high durometer such as, for example, medical grade polyurethane of high durometer. The first expandable member 20 can be formed of medical grade polyester, silicone or nylon. The second expandable member 30 can be formed of a lower durometer medical grade material such as polyurethane of lower durometer than the first expandable member 20 to have greater compliance and/or greater expandability at a lower inflation pressure(s) relative to the first expandable member 20. Other medical grade materials may be used.

Both the second and third lumen 21, 31, where used, can be attached to the same or a different fluid source, such as a fluid filled syringe(s) to allow fluid, gas and/or liquid, to move through the lumen into the respective expandable member 20, 30, to inflate and to reverse the flow to back out through the lumen back into the syringe to deflate the corresponding expandable member. In another embodiment, the second and third adjacent lumen 21, 31 can be attached at the proximal end to the same or a different rotational inflation device, such as rotational device 140 shown in FIG. 2A, to controllably inflate and deflate the respective expandable members, 20, 30.

As discussed above, the first expandable member 20 can be dilated to a specific diameter to dilate the Sphincter of Oddi, such as a diameter in a range from 1 to 20 mm or in a range from 4 to 10 mm, in some embodiments. The desired/target diameter for the inflation can vary depending on a particular patient.

In some embodiments, one or more additional lumens are attached to the catheter 10. These additional lumens can include but are not limited to having, closed or open distal ends, expandable members at distal ends, the ability to connect syringes or rotational inflation devices at proximal ends, the ability to accommodate flushing agents and contrast agents and/or low-profile devices 250 other useful characteristics.

The first and second expandable members 20, 30 can be controllably, separately (independently) inflated and deflated at different times during a treatment procedure.

FIG. 2B illustrates that the first expandable member 20 has a length L1, the second expandable member 30 has a length L2 and the first and second expandable members 20, 30 are spaced apart a longitudinal length L3. As shown, L1>L2 and L1>L3. In some embodiments, L1 is in a range of 4-20 mm, more typically in a range of 4-10 mm, such as about 5 mm, about 6 mm, about 7 mm, about 8 mm, about 9 mm, about 10 mm. In some particular embodiments, L2 can be in a range of 0.5 mm-10 mm, such as about 1 mm, about 2 mm, about 3 mm, about 4 mm, about 5 mm, about 6 mm, about 7 mm, about 8 mm, about 9 mm and about 10 mm.

When each is in a fully inflated state, the second expandable member 30 can have an increased outer diameter relative to the first expandable member 20. In some embodiments, the second expandable member 30 can expand to an outer diameter in a range of 10 mm-2 cm when fully inflated, while the first expandable member 20 can expand to an outer diameter that is 10%-400% less than the second expandable member 30, measured when each are in a fully inflated state. The desired outer diameter of each of the expandable members 20, 30 can vary depending on the target anatomy of a particular patient.

In some embodiments, the first expandable member 20 expands by 2-4× less than the second expandable member 30, with both in fully inflated operational states.

In some embodiments, the outer diameter of the second expandable member 30 can be at least 1.1×-10× greater, typically at least 50% greater, than the outer diameter of the first expandable member 20 in respective fully inflated states. For example, in some embodiments, the first expandable member 20 can be expanded to have an 8 mm outer diameter and the second expandable member 30 can be expanded to have an outer diameter of 10 mm to 2 cm.

The first expandable member 20 can have a length L1 that is 2×-4× the length of the second expandable member 30.

As shown in FIG. 2B, L2<L3. In some embodiments, L2>L3. In some embodiments L2=L3. In some embodiments, L3 can be in a range of 0.5 mm-2 cm, more typically 1 mm-15 mm, such as in a range of 1-7 mm, such as about 1 mm, about 2 mm, about 3 mm, about 4 mm, about 5 mm, about 6 mm and about 7 mm. The spacing L3 between the first and second expandable members 20, 30 can be important for manufacturability and/or proper anchoring and functionality.

Turning now to FIGS. 3-13, example actions for performing a therapeutic/interventional treatment with respect to a biliary tree are described. As is well known to those of skill in the art, the biliary tree is a system of vessels that directs these secretions from the liver, gallbladder and pancreas through a series of ducts into the duodenum. The exit hole into the duodenum is called the papilla of Vater.

FIG. 3 depicts step 1—Initial wire access into the CBD via ureteral stent.

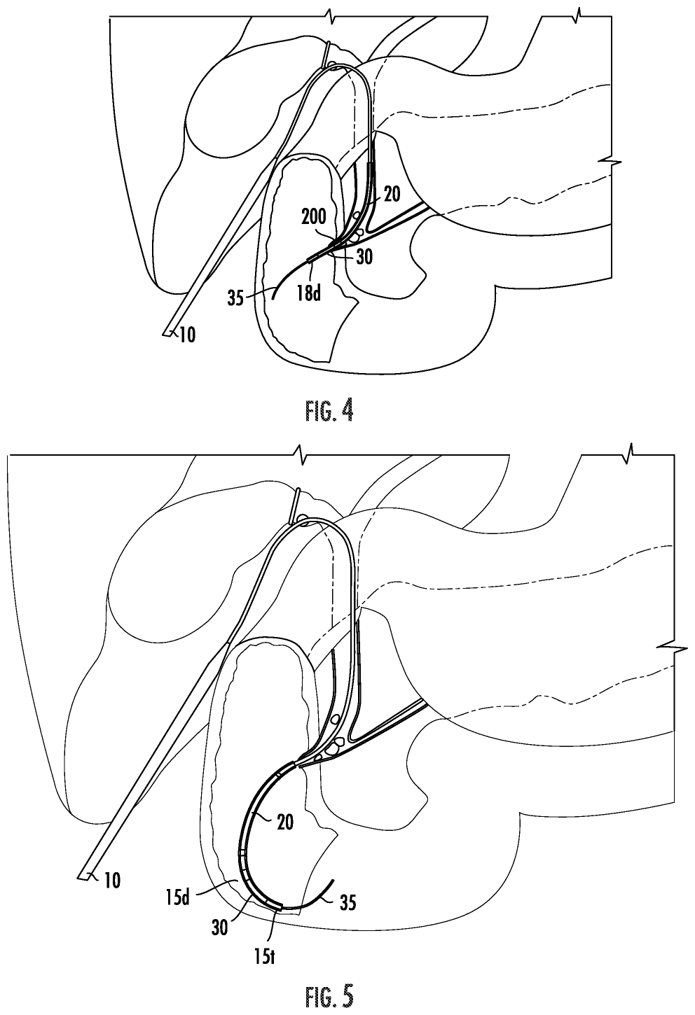

FIG. 4 depicts step 2—Advance catheter 10 across the sphincter over guidewire.

FIG. 5 depicts step 3—Catheter 10 inserted fully into the duodenum.

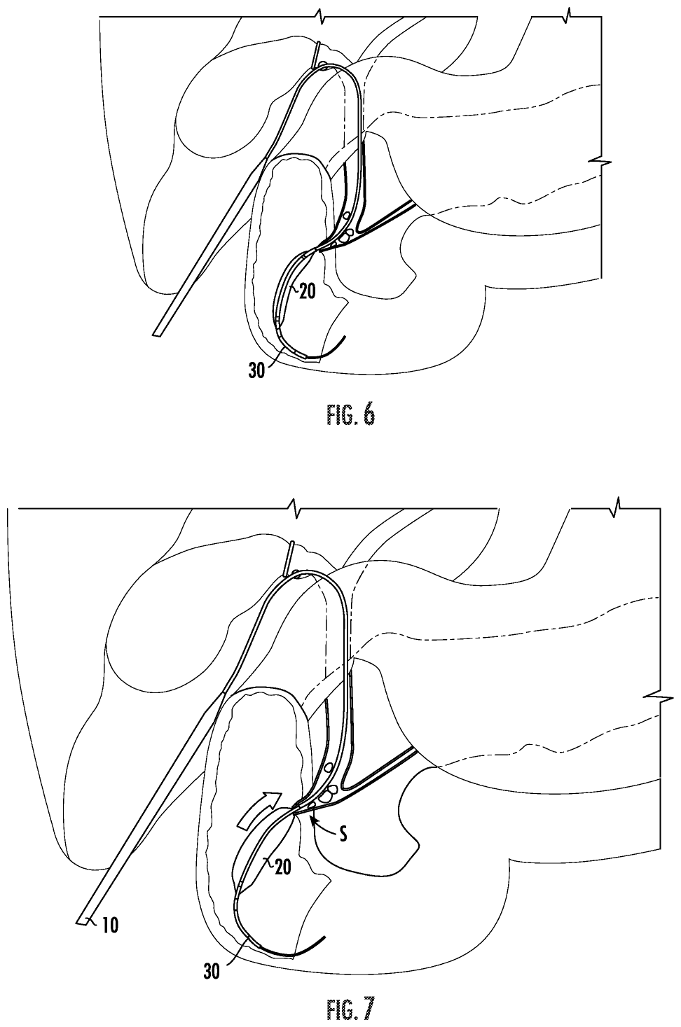

FIG. 6 depicts step 4—Noncompliant balloon 20 inflated within the duodenum.

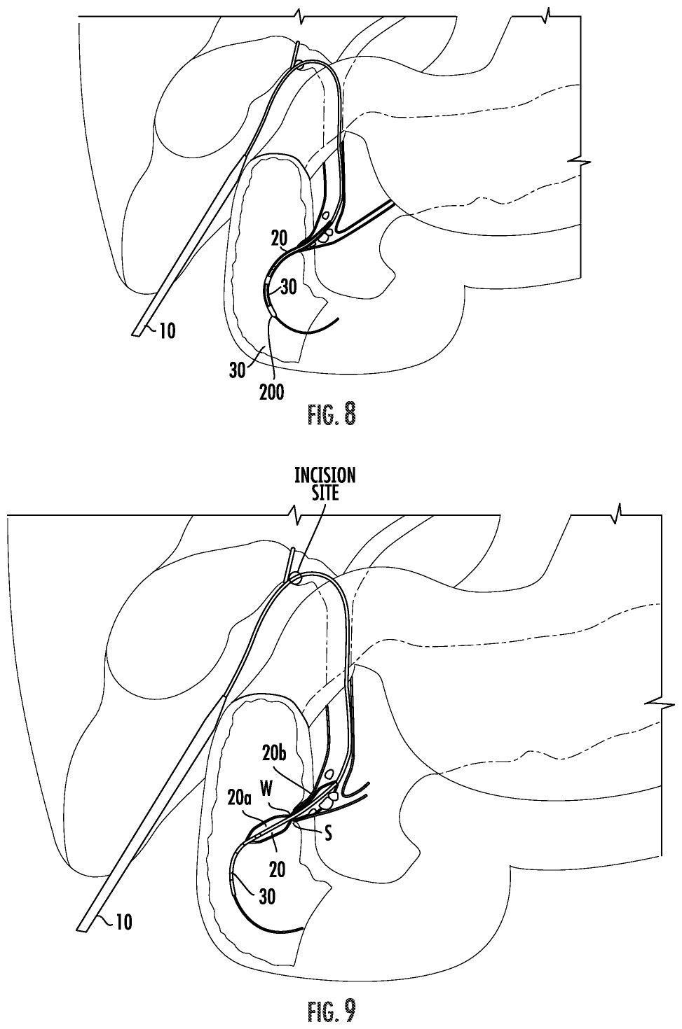

FIG. 7 depicts step 5—Noncompliant balloon 20 pulled back to access location and angle of the sphincter S.

FIG. 8 depicts step 6—Noncompliant balloon 20 deflated and then pulled back to straddle the sphincter S.

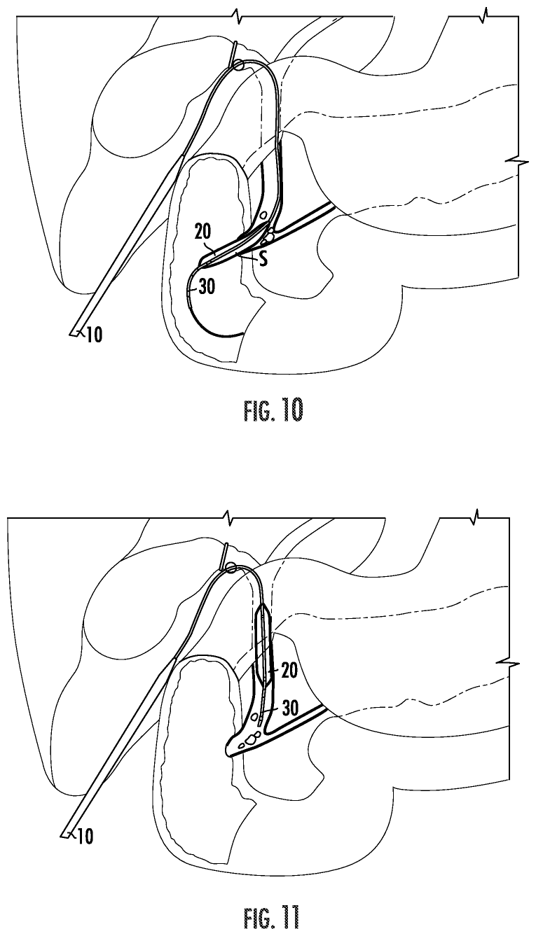

FIG. 9 depicts step 7—Noncompliant balloon 20 inflated to dilate the sphincter S—correct placement indicated by the “waist” (W) separating first and second larger segments 20a, 20b in the balloon as it comes up to profile.

FIG. 10 depicts step 8—Noncompliant balloon 20 fully inflated to dilate the sphincter S—left in position for a period of time such as several minutes, such as 2-10 minutes.

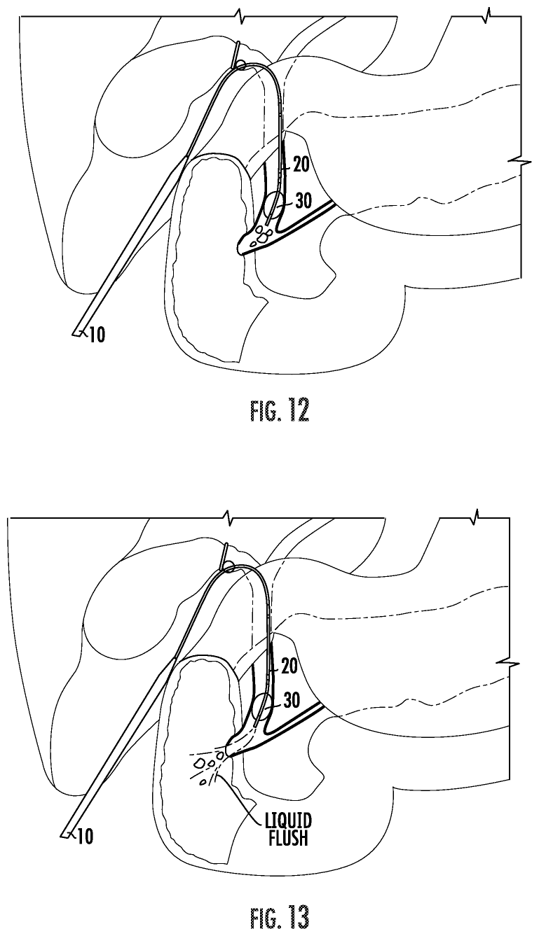

FIG. 11 depicts step 9—Noncompliant balloon 20 deflated and then pulled back into the CBD/Cystic duct.

FIG. 12 depicts step 10—The distal compliant balloon 30 is inflated to fill the lumen of the CBD and facilitate high pressure/volume flushing of the stones through the dilated sphincter.

FIG. 13 depicts step 11—High pressure, high volume flushing of the stones through the dilated sphincter S after guidewire 35 removed.

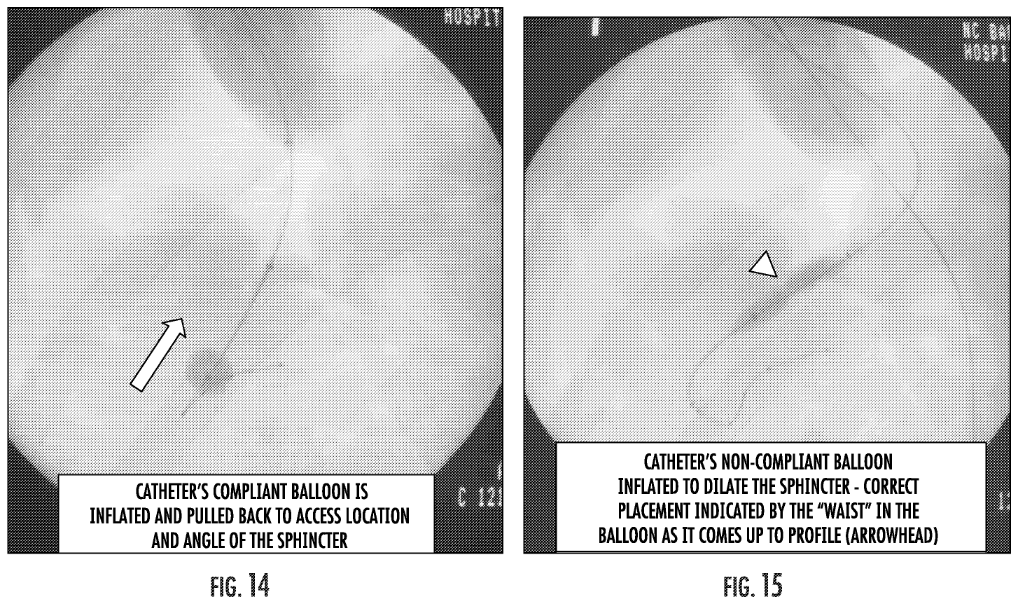

FIG. 14 depicts a fluoroscopic image demonstrating step 5—Multi-lumen catheter's second balloon (compliant balloon) 30 is inflated and pulled back to access location and angle of the sphincter.

FIG. 15 depicts a fluoroscopic image demonstrating step 7—Multi-lumen catheter's first balloon (non-compliant balloon) 20 inflated to dilate the sphincter—correct placement indicated by the “waist” in the balloon as it comes up to profile (arrowhead).

FIG. 16 depicts an X-ray image of step 10—The distal second balloon 30 (compliant balloon) (arrowhead) is inflated to fill the lumen of the Common Bile Duct and facilitate high pressure/volume flushing of the stones through the dilated sphincter.

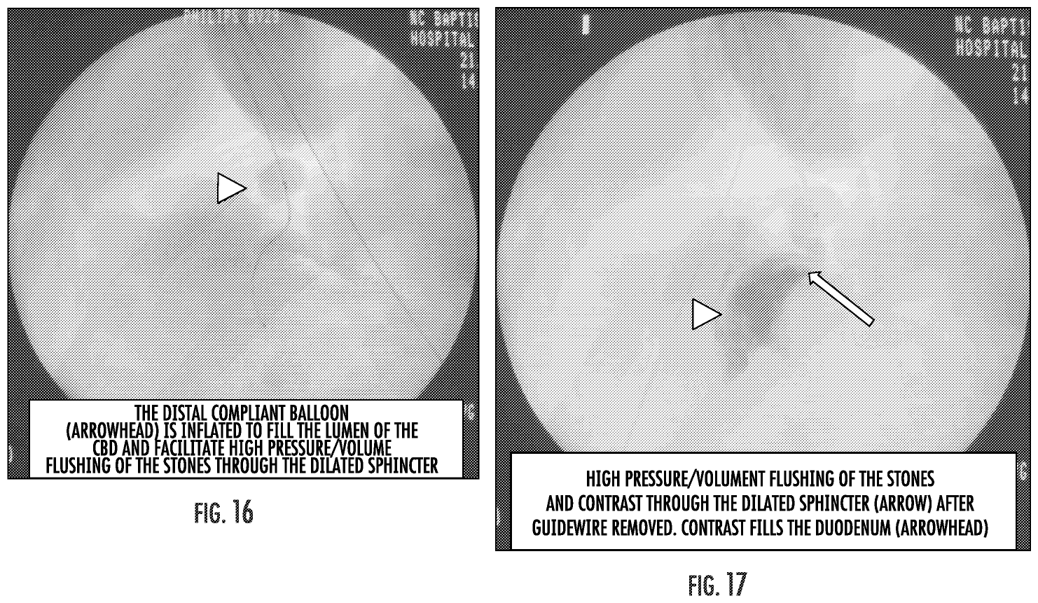

FIG. 17 depicts step 11—high pressure/volume flushing of the stones and contrast through the dilated sphincter (arrow) after guidewire 35 removed. Contrast fills the duodenum (arrowhead).

FIG. 18 schematically illustrates an interventional (low-profile) device 250 can be extended out the distal end 15d of the catheter body 15 via primary lumen 18 to retrieve/disintegrate the stones. The interventional device 250 can be provided as a wire, electrical probe, laser, ultrasound probe, or basket, by way of example.

FIG. 19 illustrates that the interventional device can comprise a nitinol basket 250′. As is well known to those of skill in the art, advancing and retracting a switch on the basket handpiece opens and closes the baskets distally. The basket 250′ can be thread through the Tuohy-Borst connector 155 and through the working channel/primary lumen 18 immediately after a filling defect is noted on intraoperative cholangiogram without any additional alterations to the setup. Nitinol baskets are available as tipped or non-tipped. The tipped baskets may be more easily visualized with fluoroscopy but may damage choledochoscopes.

Fluoroscopic images can indicate if a filling defect is present in the distal CBD without the passage of contrast into the duodenum. The second expandable member 30 can be inflated to seal the CBD. This allows for a cholangiogram to be performed through the balloon and prevents debris from being flushed proximally into the hepatic ducts. For discussions of treatments/devices, and approaches, see: Bosley M E, Zamora I J, Neff L P. Choledocholithiasis—A New Clinical Pathway. Journal of Translational Gastroenterology and Hepatology. 2021; 6:35: the contents of which are hereby incorporated by reference as if recited in full herein.

The back aperture of the Tuohy-Borst connector 155 (FIG. 2A) can be closed when performing a cholangiogram and then subsequently opened to accept a (0.035 inch) guidewire 35 as needed. In order to successfully thread the wire into the system, the catheter 10 can be backed out of the Tuohy-Borst connector 155. The connector 155 can also be closed-down on the wire once it is in position. This configuration can facilitate a “step-up approach” to dilation of the cystic duct with the non-compliant balloon to create sufficient cystic duct luminal diameter for introduction of other devices such as endoscopes, laparoscopic common bile duct exploration and balloon sphincteroplasty with the next step immediately available to the operator using the same platform. Maximizing the likelihood for success for endoscopic or catheter-based interventions such as balloon sphincteroplasty not only includes a system through which to perform the procedure, but also consideration of operating room ergonomics.

When performing the procedure, one or more monitors in the operating room(s) can be configured to display both the fluoroscopy and laparoscopy images simultaneously. While seemingly a minor point, optimization of the OR (operating room) layout can allow for more favorable working conditions and efficiency. Successful management of the wire and catheters can optionally be facilitated by a team of three people. It can be helpful to have one person maintain forward tension on the catheter 10 while the primary surgeon manages the flushing and catheter manipulation, and a third person manages the wires and equipment on a back table.

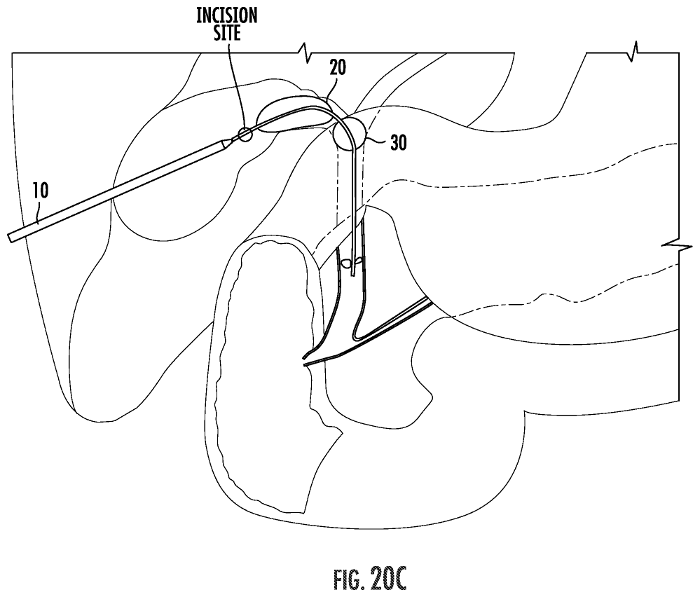

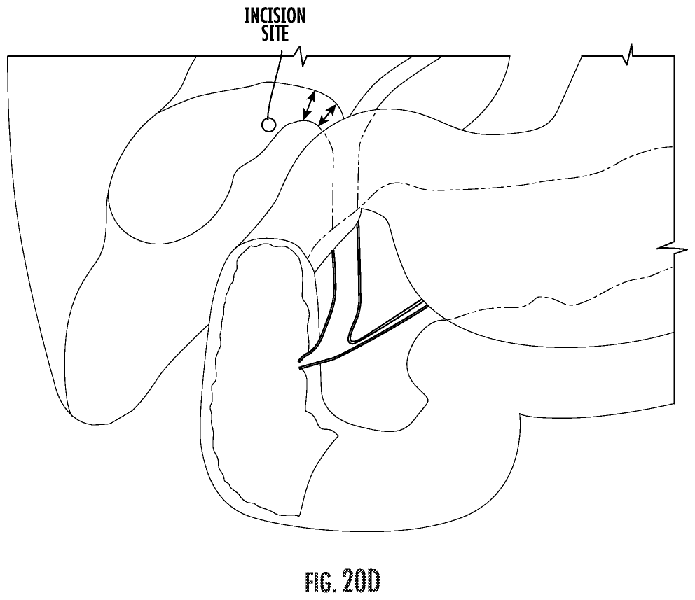

Turning now to FIGS. 20A-20D, example actions for performing an alternative workflow for a therapeutic/interventional treatment with respect to a biliary tree are described. FIG. 20A depicts an incision site I, similar to that shown with respect to FIG. 3, for use in a ductotomy procedure to remove one or more stones (Stones) in the biliary tree without requiring dilation of a sphincter.

FIG. 20B depicts that the catheter 10 can be inserted via the incision site I through the cystic duct (CD) over a previously placed guidewire 35 so that the second expandable member 30 resides in the primary biliary channel where it is then inflated to anchor the catheter in position while the first expandable member 20 remains in the CD.

FIG. 20C depicts the second expandable member 30 remains expanded in the primary biliary channel with the first expandable member 20 is then inflated in the CD to dilate the CD (FIG. 20D) in preparation for insertion of a choledochoscope or other medical device.

FIG. 20D illustrates that the catheter 10 can be removed with the CD having been dilated downstream of the incision site I for passage of the choledochoscope.

Embodiments of the invention will be described further below by way of the following non-limiting Examples.

### Examples

### Procedure

During an example procedure, the primary or central lumen of the catheter 10 is advanced over a guidewire 35 previously placed through a transductal opening in the biliary tree and distally terminating at any length along its path into the duodenum. The first expandable member 20 is inflated and pulled back to access location and angle of a sphincter. In other embodiments, any expandable member can access location and angle of a sphincter S. Next, the first expandable member 20 is deflated and pulled back to traverse the sphincter. Once the first expandable member 20 is across the sphincter, it is inflated to stretch sphincter. In some embodiments, the first expandable member 20 is left in place for several minutes. This is done so the tissue briefly maintains a preferred shape and form. Once stretching of the tissue is complete, the first expandable member 20 is deflated and the catheter 10 is pulled back proximally within the biliary tree. The second expandable member 30 is inflated in the biliary tree after removing the guidewire 35. While the second expandable member 30 remains inflated, the biliary tree is flushed through the primary or central lumen 18 with saline and/or contrast. The first expandable member 20 can remain at least partially, typically totally deflated so as to reside at a common profile with adjacent segments of the catheter body when the second expandable member 30 is inflated and sealed against local tissue. The first expandable member 20 can be upstream of the second expandable member and can be in the biliary tree or outside the biliary tree, even potentially outside a cut of the cystic duct. In some embodiments, one or more low-profile medical devices pass through the primary lumen 18, such as an interventional wire or a stone retrieval basket.

### Methods

The catheter 10 (FIGS. 1A, 2A) allows for a cholangiogram to be performed through the lumen of the catheter for the identification of common bile duct stones and delineation of the biliary tree anatomy. The advantage of this system is that the distal tip of the catheter has a round (spherical) balloon 30 which can be inflated to seal and then pressurize the bile duct distally when performing the cholangiogram and for flushing stones forward into the duodenum before or after dilation of the Sphincter of Oddi. If pressurized flushing is unsuccessful, the distal balloon can be deflated and the elongated proximal balloon 20 can be advanced to straddle the sphincter. Once the second balloon is in position, it can be inflated to dilate the sphincter which should then facilitate subsequent attempts for forward flushing of the retained stones. For example, after dilation, the smaller balloon 30 can once again be utilized to seal the duct and flush stones forward into the duodenum. All of these steps can be performed sequentially without the need for device exchanges.

The distal balloon can also be inflated just inside the common bile duct to anchor the catheter at the junction of the cystic duct and common bile duct. This will facilitate positioning and effective inflation of the non-compliant balloon to dilate the cystic duct to enlarge its diameter and permit insertion of other devices such as endoscopes.

The device shown in FIG. 1A was tested in a model in which peanuts were inserted into the common bile duct to act as common bile duct stones. Clearance of the duct was attempted with the novel device and was successful.

### Results

Initially fluoroscopy demonstrated the “stones” (peanuts) in the common bile duct of the animal. The common bile duct was sealed with the distal balloon and a subsequent cholangiogram performed with pressurized flushing. The sphincter was dilated by the more proximal elongated balloon. After dilation the proximal balloon was deflated and the catheter withdrawn. The distal balloon re-created a seal for final cholangiogram/power flushing. The “stones” were noted to have advanced forward into the duodenum (FIG. 17).

### CONCLUSIONS

The balloon occlusion cholangiography and sphincteroplasty catheter is a novel device that allows for performing a cholangiogram, dilation of the sphincter, and pressurized flushing of the CBD without requiring any instrument changes and with a single intrabody insertion to place the multi-lumen catheter. It has been determined to be successful in clearance of common bile duct stones in an animal model and may prove to be helpful in an operative setting. The multi-lumen catheter can provide a double biliary balloon dilator for laparoscopic common bile duct exploration (lap CBDE).

Choledocholithiasis can be managed safely by either laparoscopic cholecystectomy (LC) with pre-/post-operative endoscopic retrograde cholangiopancreatography (ERCP) or LC with intraoperative laparoscopic common bile duct exploration (LCBDE). LC+LCBDE has the advantage of being a one-step, one-anesthetic process. This technique has decreased in utilization over the past decade in the United States.

The multi-lumen catheter 10 (such as shown in FIG. 1A) allows for a cholangiogram to be performed through the lumen of the catheter for the identification of common bile duct stones. The advantage of this system is that the distal tip of the catheter has a (round) balloon 30 which can be inflated to seal and then pressurize the bile duct distally when performing the cholangiogram and attempting to flush stones forward. If pressurized flushing is unsuccessful, the distal balloon can be deflated and the elongated proximal balloon can be advanced to straddle the sphincter. Once the second balloon 20 is in position, it can be inflated to dilate the sphincter which then should allow for forward flushing of the retained stones. After dilation, the smaller proximal balloon can once again be utilized to seal the duct and flush stones forward into the duodenum. All of these steps can be performed sequentially without the need for any device exchange. This prototype device was tested in a pig animal model in which peanuts were inserted into the common bile duct to act as common bile duct stones. Clearance of the duct was attempted with the novel device.

In the drawings and specification, there have been disclosed embodiments of the invention and, although specific terms are employed, they are used in a generic and descriptive sense only and not for purposes of limitation, the scope of the invention being set forth in the following claims.

Thus, the foregoing is illustrative of the present invention and is not to be construed as limiting thereof. More particularly, the workflow steps may be carried out in a different manner, in a different order and/or with other workflow steps or may omit some or replace some workflow steps with other steps. Although a few exemplary embodiments of this invention have been described, those skilled in the art will readily appreciate that many modifications are possible in the exemplary embodiments without materially departing from the novel teachings and advantages of this invention.

Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

### REFERENCES (INCORPORATED BY REFERENCE AS IF RECITED IN FULL HEREIN)

1. McNicoll C F, Pastorino A, Farooq U, et al. Choledocholithiasis. [Updated 2021 Aug. 31]. In: StatPearls [Internet]. Treasure Island (FL): StatPearls Publishing; 2021 January. Available from: https://www.ncbi.nlm.nih.gov/books/NBK441961/
2. Legorreta A P, Silber J H, Costantino G N, Kobylinski R W, Zatz S L. Increased Cholecystectomy Rate After the Introduction of Laparoscopic Cholecystectomy. JAMA. 1993; 270(12):1429-1432. doi:10.1001/jama.1993.03510120051029
3. Urbach D R. Rate of elective cholecystectomy and the incidence of severe gallstone disease. Can Med Assoc J. 2005; 172(8):1015-1019. doi:10.1503/cmaj.1041363
4. Bonfrate L, Wang D Q-H, Garruti G, Portincasa P. Obesity and the risk and prognosis of gallstone disease and pancreatitis. Best Pract Res Clin Gastroenterol. 2014; 28(4):623-635. doi:10.1016/j.bpg.2014.07.013
5. Hungness E S, Soper N J. Management of common bile duct stones. J Gastroinstest Surg. 2006; 10(4):612-619. doi:10.1016/j.gassur.2005.08.015
6. Hunter J G, Soper N J. Laparoscopic management of bile duct stones. Surg Clin North Am. 1992; 72(2):1077
7. Fizgibbons Jr Robert J, Gardner G C. Laparoscopic Surgeon and the Common Bile Duct. World J Surg. 2001; 25(10):1317-1324. doi:10.1007/s00268-001-0117-1
8. Maple J T, Ben-Menachem T, Anderson M A, et al. The role of endoscopy in the evaluation of suspected choledocholithiasis. Gastrointest Endosc. 2010; 71(1):1-9. doi:10.1016/j.gie.2009.09.041
9. Dasari B V M, Tan C J, Gurusamy K S, et al. Surgincal versus endoscopic treatment of bile duct stones. Cochrane Database Syst Rev. 2013; (12):CD003327.pub4
10. Bansal V K, Misra M C, Rajan K, et al. Single-stage laparoscopic common bile duct exploration and cholecystectomy versus two-stage endoscopic stone extraction followed by laparoscopic cholecystectomy for patients with concomitant gallbladder stones and common bile duct stones: a randomized controlled trail. Surg Endosc. 2014; 28(3):875-885. doi:10.1007/s00464-013-3237-4
11. Cuschieri A, Lezoche E, Morino M, et al. E.A.E.S. multicenter prospective randomized trial comparing two-stage vs single-stage management of patients with gallstone disease and ductal calculi. Surg Endosc. 1999; 13(10):952-957. doi:10.1007/s004649901145
12. Wandling M W, Hungness E S, Pavey E S, et al. Nationwide Assessment of Trends in Choledocholithiasis Management in the United States From 1998 to 2013. JAMA Surg. 2016; 151(12):1125-1130. doi: 10.1001/jamasurg.2016.2059
13. Topal B, Vromman K, Aerts R, Verslype C, Van Steenbergen W, Penninckx F. Hospital cost categories of one-stage versus two-stage management of common bile duct stones. Surg Endosc. 2010; 24(2):413-416. doi:10.1007/s00464-009-0594-0
14. Mattila A, Mrena J, Kellokumpu I. Cost-analysis and effectiveness of one-stage laparoscopic versus two-stage endolaparoscopic management of cholecystocholedocholithiasis: a retrospective cohort study. BMC Surg. 2017; 17(1):79. doi:10.1186/s12893-017-0274-2

1. A multi-lumen catheter comprising:
a catheter body comprising opposing proximal and distal end portions;
a first expandable member on the distal end portion;
a second expandable member on the distal end portion, longitudinally spaced apart from the first expandable member;
a primary lumen that extends longitudinally and that is open at opposing proximal and distal ends thereof, wherein the distal end of the primary lumen resides distally of the first and second expandable members; and
at least one additional lumen in fluid communication with the first and second expandable members to controllably inflate and deflate the first and second expandable members.

2. The multi-lumen catheter of claim 1, wherein the second expandable member is distal of the first expandable member and has a longitudinally extending length that is less than a length of the first expandable member.

3. The multi-lumen catheter of claim 1, wherein the first expandable member comprises a material with a first durometer, wherein the second expandable member comprises a material with a second durometer, wherein the first durometer is greater than the second durometer whereby the first expandable member is configured to have less flexibility than the second expandable member thereby providing a non-compliant body suitable for dilating a sphincter and/or other target tissue.

4. The multi-lumen catheter of claim 1, wherein the first expandable member has a fully inflated state defining an outer diameter, wherein the second expandable member has a fully inflated state defining an outer diameter, and wherein the outer diameter of the first expandable member is less than the outer diameter of the second expandable member in respective fully inflated states.

5. The multi-lumen catheter of claim 1, wherein a length of the first expandable member is 2-4 times greater than a length of the second expandable member.

6. The multi-lumen catheter of claim 1, wherein the first expandable member is closely spaced apart from the second expandable member by a longitudinal distance that is in a range of 0.5 mm-20 mm.

7. The multi-lumen catheter of claim 1, further comprising valves upstream of the first and second expandable members in fluid communication with the at least one additional lumen, wherein the first and second expandable members are independently/separately inflatable and deflatable.

8. The multi-lumen catheter of claim 1, wherein the catheter body is sized and configured for placement through a transductal opening in a biliary tree and the distal end portion is sized and configured to distally terminate and be able to slidably extend into and retract from a duodenum of a patient.

9. The multi-lumen catheter of claim 1, wherein the primary lumen is coupled to a syringe comprising liquid and is configured to allow liquid to pass through the distal end of the primary lumen into a duct of a patient, and wherein the primary lumen is configured to allow a medical device to be deployed through the primary lumen into the duct of the patient.

10-15. (canceled)

16. The multi-lumen catheter of claim 1, further comprising a plurality of longitudinally spaced apart radiopaque markings positioned along the distal end portion of the catheter body thereby allowing visual indication of a location of one or both of the first and second expandable members in a medical image.

17. The multi-lumen catheter of claim 1, wherein the first expandable member has an elongate shape and is made of a semi-rigid material, and wherein the second expandable member has a spherical shape when inflated and is formed of a material with increased flexibility relative to the first expandable member.

18. The multi-lumen catheter of claim 1, wherein the first expandable member is configured to expand to a diameter sufficient to dilate a target sphincter, and wherein the diameter is in a range of 1 to 20 mm.

19-20. (canceled)

21. The multi-lumen catheter of claim 1, further comprising a rotational inflation device coupled to a first lumen provided by the at least one additional lumen, wherein the first lumen is in fluid communication with the first and/or second expandable member.

22-26. (canceled)

27. A method for using the multi-lumen catheter of claim 1 comprising:
a) advancing the multi-lumen catheter over a previously placed guidewire through a formed or natural orifice in a body of a patient toward a duodenum;
b) at least partially inflating the first expandable member when the first expandable member is past a sphincter and in the duodenum;
c) at least partially deflating and pulling back the first expandable member to traverse the sphincter;
d) inflating the first expandable member to stretch the sphincter while the first expandable member straddles the sphincter; then
e) deflating the first expandable member and pulling the multi-lumen catheter and guidewire together back in a proximal direction;
f) inflating the second expandable member to form a barrier in a duct; and
g) discharging liquid from the multi-lumen catheter to flush debris in a distal direction out of a duct of a biliary tree.

28. (canceled)

29. The method of claim 27, wherein the inflation of the second expandable member to form the barrier in the duct is within the biliary tree, and wherein the first expandable member is at least partially deflated and resides outside the biliary tree or in the biliary tree when the second expandable member forms the barrier in the duct.

30-34. (canceled)

35. A biliary treatment system, comprising:
a multi-lumen catheter comprising first and second expandable members on a distal end portion thereof, wherein the multi-lumen catheter comprises a primary lumen and at least one additional lumen residing adjacent thereto and extending longitudinally, wherein the distal end portion comprises radiopaque markers;
a syringe comprising liquid coupled to the multi-lumen catheter and in fluid communication with the first and/or second expandable members and configured to inflate and deflate the first and/or second expandable members; and
a Touhy-Borst connector coupled to the multi-lumen catheter and in communication with the primary lumen.

36. The biliary treatment system of claim 35, wherein the syringe is in fluid communication with only the second expandable member, the biliary treatment system further comprising a rotational inflation device in fluid communication with the first expandable member to controllably inflate and deflate the first expandable member.

37. The biliary treatment system of claim 35, further comprising a guidewire configured to be slidably received in the primary lumen.

38. The biliary treatment system of claim 35, wherein the second expandable member is distal of the first expandable member and has a longitudinally extending length that is less than a length of the first expandable member.

39. The biliary treatment system of claim 35, wherein the first expandable member comprises a material with a first durometer, wherein the second expandable member comprises a material with a second durometer, wherein the first durometer is greater than the second durometer whereby the first expandable member is configured to have less flexibility than the second expandable member thereby providing a non-compliant body suitable for dilating a cystic duct and/or a sphincter.

40. The biliary treatment system of claim 35, wherein the first expandable member has a fully inflated state defining an outer diameter, wherein the second expandable member has a fully inflated state defining an outer diameter, and wherein the outer diameter of the second expandable member is at least 50% greater than the outer diameter of the second first expandable member in respective fully inflated states.

41. The biliary treatment system of claim 35, wherein a length of the first expandable member is 2-4 times greater than a length of the second expandable member.

42. The biliary treatment system of claim 35, wherein the first expandable member is closely spaced apart from the second expandable member by a longitudinal distance that is in a range of 0.5-20 mm.

43. The biliary treatment system of claim 35, further comprising valves upstream of the first and second expandable members in fluid communication with the at least one additional lumen, wherein the first and second expandable members are independently/separately inflatable and deflatable.

44. The biliary treatment system of claim 35, wherein the first expandable member has an elongate shape and is made of a semi-rigid material, and wherein the second expandable member has a spherical shape when inflated and is formed of a material with increased flexibility relative to the first expandable member.

45. The biliary treatment system of claim 35, further comprising a medical device sized and configured to slidably deploy from a distal end of the multi-lumen catheter.

46. The biliary treatment system of claim 35, wherein the first expandable member is configured to dilate a sphincter in response to inflation pressure in a range of 1 atm to 10 atm to expand to an inflated state, and wherein the second expandable member is configured to seal against walls of a duct in an inflated state having a larger outer diameter in response to inflation pressure that is lower than the inflation pressure applied to the first expandable member to inflate to one or more of its inflated operational inflated diameters and while the first expandable member is in at least a partially deflated state.

47. A method for using the multi-lumen catheter of claim 1 comprising:
a) advancing the multi-lumen catheter over a previously placed guidewire through a formed or natural orifice in a body of a patient toward a duodenum;
b) inflating the second expandable member to form a barrier in a duct in a biliary tree downstream of a cystic duct to thereby anchor the first inflatable member in the cystic duct; and
c) inflating the first expandable member in the cystic duct to dilate the cystic duct.

48. The method of claim 47, further comprising deflating the first and second expandable members, separately or concurrently, then withdrawing the catheter from the cystic duct, then inserting a choledochoscope into the dilated cystic duct to perform a medical procedure to thereby remove a stone or stones from the biliary tree.
